(19) United States
(12) Patent Application Publication
(10) Pub. No.: US 2026/0232690 A1
(43) Pub. Date: Aug. 13, 2026

(54) Treatment of the Chronic Effects of Brain Injury Using Rho Kinase Inhibitors

(71) Applicant: WOOLSEY PHARMACEUTICALS, INC., Petersburg, FL (US)

(72) Inventors: Thomas MacAllister, Arlington, VA (US); Sven Jacobson, East Hampton, NY (US)

(73) Assignee: WOOLSEY PHARMACEUTICALS, INC., Petersburg, FL (US)

(21) Appl. No.: 19/150,613

(22) PCT Filed: Jan. 25, 2024
(86) PCT No.: PCT/US24/12891

§ 371 Date: Jul. 24, 2025

Related U.S. Application Data
(60) Provisional application No. 63/481,806, filed on Jan. 27, 2023.

(51) Int. Cl. A61K 31/551; A61P 25/00

(52) U.S. Cl. CPC A61K 31/551; A61P 25/00

(57) ABSTRACT

Provided is a method for treating chronic effects of a brain injury, including stroke, using fasudil. The method comprises treating a patient beginning more than three months following the brain injury.

### BACKGROUND

The present invention relates to methods and agents useful for the treatment of brain injuries, which may be the result of endogenous or exogenous injury. Endogenous injury typically results from a stroke, including ischemic stroke and hemorrhagic stroke, or from a hereditary and/or congenital condition. Exogenous injury, including concussion and other traumatic brain injuries (TBIs), typically results from acceleration and/or deceleration injury that may or may not result in the head coming into contact with a hard surface. In particular, compositions and methods for treating the chronic effects of a brain injury in a subject are provided, beginning more than three months following the occurrence of the brain injury.

Brain injury is any injury to the brain that results in abnormal brain function; a brain injury may be hereditary, congenital or acquired. Acquired brain injuries include traumatic and acute non-traumatic forms that can result from a wide range of forces and factors that affect the brain including falls, accidents, sports activities, strikes by objects, explosions and other traumatic events as well as stroke, anoxia, hypoxia, toxins, infections, cancer and other internal and external factors.

TBI is a form of acquired brain injury that refers to damage to the brain resulting from external mechanical force. TBI can result from falls, firearm wounds, sports accidents, construction accidents and vehicle accidents, among other causes. TBI is a major cause of death, disability, and mental health disorders. It is the leading cause of disability and death in people under 45 with approximately 10 million new cases each year worldwide.

TBI patients can suffer from a number of physical, cognitive, social, emotional and/or behavioral disorders following injury, which may be long-lasting or even permanent. According to the diagnostic criteria detailed in the “Diagnostic and Statistical Manual of Mental Disorders (DSM-5) TBI has one or more of the following characteristics: changes in levels of consciousness; memory disturbances; confusion associated with deficits in orientation; neurological signs, such as brain injury observable on neuroimaging, new onset or worsening of seizure disorder, visual field deficits and hemiparesis.

The acute treatment of brain injury is based on the severity of the injury and can range from no treatment, or simple rest with monitoring, to immediate emergency and follow up surgical care to maintain adequate blood pressure, blood supply and oxygen to the brain. Treatment may also include rehabilitative therapy and providing medications to limit secondary damage to the brain due to inflammation, cellular instability, hemorrhage, and reduced oxygen supply. Medications that are often used to limit secondary damage immediately after an injury include anti-inflammatory drugs, diuretics, anticoagulants, anticonvulsants, muscle relaxants and coma-inducing drugs. The chronic treatment of brain injury focuses more on rehabilitation and symptomatic treatments.

Stroke. Stroke, an acquired form of brain injury resulting from endogenous injury, remains the third most common cause of death in the industrialized world behind heart disease and cancer. Every year, 15 million people throughout the world suffer a stroke, and five million are left significantly disabled. There are two forms of stroke: ischemic stroke and hemorrhagic stroke. Ischemic stroke or cerebral ischemia is caused by a temporary or permanent restriction of cerebral blood flow and oxygen supply caused by, e.g., an embolis (embolic stroke) or blood clot (thrombolytic stroke). A hemorrhagic stroke is caused by a blood vessel rupture (e.g., ruptured aneurysm), which leads to severe bleeding in the brain. There are also certain genetic predispositions to stroke that are caused by thickening of arteries. Ischemic stroke accounts for approximately 80-86% of all stroke cases.

Ischemic stroke is treated with emergency endovascular procedures or anti-clotting therapeutics. Current pharmacotherapy for ischemic stroke is limited. The main treatment available for acute treatment of ischemic stroke rTPA, a thrombolytic agent. Anti-thrombolytic agents must be administered within a short window following stroke onset, and do not address long-term consequences following a stroke and so their usefulness is limited. It carries an increased risk of intracranial hemorrhage, reperfusion injury, and diminishing cerebral artery reactivity. Thus, rTPA is not used to treat hemorrhagic stroke and is contraindicated in certain types of stroke such as subarachnoid hemorrhage. Treatments for hemorrhagic stroke are even more limited, and include surgery, endovascular embolization, and cessation of drugs that cause bleeding such as warfarin.

Recovering from a stroke takes months to years and frequently requires intensive rehabilitation. The immediate clinical consequences of the stroke are subsequently complicated by a variety of medical, musculoskeletal and psychosocial difficulties. Common types of disabilities following a stroke include impaired speech, restricted physical abilities, weakness or paralysis of limbs on one side of the body, difficulty gripping or holding things, impaired visuo-perceptual skill, dysphagia, mood, behavior, and personality changes, impaired cognition, and impaired memory formation. The disabilities can be permanent, depending on the type of stroke, the area of the brain affected, and how long the brain was deprived of oxygen. While the initial recovery rate up to three months is rapid, between three and six months after stroke recovery is significantly slowed. (Lee 2015).

Further, prior stroke is a significant risk factor for the development of further strokes. Patients who have had a stroke are about four times more likely to have another stroke than matched controls.

Accordingly, there is a need for effective pharmacological treatment of the chronic effects of brain injury and secondary prevention of brain injury caused by further strokes or to prevent further complications following a brain injury.

### SUMMARY

Disclosed is a method of treating a patient suffering from chronic effects of brain injury, comprising orally administering to said patient a therapeutically effective amount of a fasudil, hydroxyfasudil, or a pharmaceutically acceptable salt, hydrophobic ion pair or hydrate thereof.

In one embodiment, the therapeutically effective amount exceeds 60 mg per patient per day.

In another embodiment, the brain injury occurred more than three months prior to initiating treatment.

In another embodiment, fasudil hydrochloride hemihydrate, or molar equivalent thereof, is administered.

In a further embodiment to the foregoing, the brain injury is a traumatic brain injury.

In one embodiment, the traumatic brain injury resulted from a head contacting a hard surface.

In one embodiment, the patient exhibits cognitive deficits.

In another embodiment, the brain injury is a stroke.

In a further embodiment, the stroke is a hemorrhagic stroke.

In yet a further embodiment the stroke is a cerebral stroke, or a cortical stroke.

In yet another embodiment, the stroke is a sub-cortical stroke.

In one embodiment, the cortical stroke is a cerebellar stroke.

In a further embodiment, the hemorrhagic stroke is a large vessel stroke.

In another embodiment, the stroke affects the middle cerebral artery (MCA), the internal carotid artery (ICA), the posterior Inferior cerebellar artery (PICA), and the superior cerebellar artery (SCA).

In a further embodiment, the stroke is a small vessel stroke.

In further embodiment, the hemorrhagic stroke is selected from the group consisting of epidural hematoma, subdural hematoma, subarachnoid hemorrhage (SAH), intraventricular hemorrhage (IVH), hemorrhagic transformation of ischemic stroke (HT), venous hemorrhage from cortical vein or sinus thrombosis, and intracerebral hemorrhage (ICH).

In yet another embodiment, the hemorrhagic stroke is a lacunar stroke.

In yet another embodiment, the stroke is not a sub-cortical stroke or a stroke affecting white matter.

In a specific embodiment, the stroke is not a lacunar stroke.

In a further embodiment, the brain injury patient does not have Binswanger's disease.

In one embodiment, the brain injury patient does not have hypertension or coronary artery disease.

In another embodiment, the brain injury patient does not have dementia.

In one embodiment, fasudil hydrochloride hemihydrate is administered to the brain injury patient at a total dose of 70 to 240 mg per patient per day.

In another embodiment, the fasudil hydrochloride hemihydrate is administered to the patient at a total dose of 90 to 180 mg per patient per day.

### DETAILED DESCRIPTION

The present disclosure contemplates treating patients suffering from the chronic effects of a brain injury with a ROCK inhibitor such as fasudil or a salt or hydrate thereof. Patients treatable according to the invention will have gone through a period of recovery from a brain injury, after which their recovery plateaus and stabilizes, relative to the acute phase. Generally, this occurs more than three months after the brain injury and, commonly, may not occur until six or twelve months following the brain injury. Accordingly, patients treatable according to the invention will have suffered their brain injury more than three months prior to initiating treatment. In some cases, treatment will be initiated more than six or more than nine or even more than twelve months following the brain injury.

Fasudil. Fasudil (5-(1,4-Diazepane-1-sulfonyl) isoquinoline) is an isoquinoline rho kinase (ROCK) inhibitor developed by the Asahi Chemical Industry (Tokyo, Japan). Fasudil has been approved in Asia for the treatment of cerebral vasospasm and glaucoma.

Two mammalian ROCK homologs are known, ROCK1 (aka ROKβ, Rho-kinase β, or p160ROCK) and ROCK2 (aka ROKα). In humans, the genes for both ROCK1 and ROCK2 are located on chromosome 18. The two ROCK isoforms share 64% identity in their primary amino acid sequence, whereas the homology in the kinase domain is even higher (92%). Both ROCK isoforms are serine/threonine kinases and have a similar structure. Leukocyte ROCK activity has been reported to be elevated in patients following ischemic stroke, which was a prognostic indicator of a recurring stroke, along with plasma high-sensitivity C-reactive protein (Cheng 2014).

Fasudil is a potent, selective inhibitor of both ROCK isoforms, and also inhibits other protein kinases such as PKC and MLCK. In vivo, fasudil is subjected to hepatic metabolism to its active metabolite hydroxyfasudil (aka, M3). M3 is likely responsible for the bulk of the in vivo pharmacological activity of the molecule. Treatment results in a potent relaxation of vascular smooth muscle, resulting in enhanced blood flow (Shibuya 2001). A particularly important mediator of vasospasm, ROCK induces vasoconstriction by phosphorylating the myosin-binding subunit of myosin light chain (MLC) phosphatase, thus decreasing MLC phosphatase activity and enhancing vascular smooth muscle contraction. Moreover, there is evidence that fasudil increases endothelial nitric oxide synthase (eNOS) expression by stabilizing eNOS mRNA, which contributes to an increase in the level of the potent vasodilator nitric oxide (NO), thereby enhancing vasodilation. (Chen 2013).

Fasudil has been approved in China and Japan for treating cerebral vasospasm following a subarachnoid hemorrhage.

Kamei (1996a-b) reported on using fasudil for a few weeks in two patients with wandering due to vascular dementia. One patient was diagnosed with Binswanger-type cerebral infarction. The other patient was diagnosed with sequelae of cerebral bleeding and multiple lacunar infarctions, confirmed by MRI. Fasudil also has been evaluated in salt-loaded hypertensive stroke-prone rats and shown to attenuate nephrosclerosis and improve survival. (Nishikimi 2007). Both patients were taking numerous other drugs including pentoxyfilline, propentoxyfilline, moxisylyte, vinpocetine, idebenone which likely confounded the results. Pentoxyfilline, proentoxyfilline, moxisylyte, and vinpocetine all affect blood flow.

As used herein fasudil can be in the form of a hydrate such as a hemihydrate, shown below.

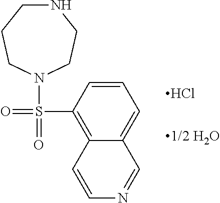 [SMILES: O=S(=O)(c1cccc2cnccc12)N1CCCNCC1 ; "·1/2 H2O" ; Cl]

Other examples of isoquinoline derived ROCK inhibitors include dimethylfasudil and ripasudil. Still other ROCK inhibitors are based on a 4-aminopyridine structure. (Feng 2015).

### Methods of Treatment

The present invention relates to the use of fasudil to treat the chronic effects of a brain injury. Brain injury patients treatable according to the invention will have suffered their brain injury more than three months prior to the initiation of treatment. Brain injuries treatable according to the invention may be the result of endogenous or exogenous injury. Endogenous injury typically results from a stroke, including ischemic stroke and hemorrhagic stroke, or from a hereditary and/or congenital condition. Exogenous injury, including concussion and other traumatic brain injuries (TBIs), typically results from acceleration and/or deceleration injury that may or may not result in the head coming into contact with a hard surface.

As used herein, a “brain injury” is any injury to the brain that results in abnormal brain function; a brain injury may be hereditary, congenital or acquired. Acquired brain injuries include traumatic and acute non-traumatic forms that can result from a wide range of forces and factors that affect the brain including falls, accidents, sports activities, strikes by objects, explosions and other traumatic events as well as stroke, anoxia, hypoxia, toxins, infections, cancer and other internal and external factors. The term brain injury specifically contemplates traumatic brain injury and stroke.

In one embodiment, the brain injury results from participation in sports.

In another embodiment, the brain injury results from an accident.

In a further embodiment, the brain injury results from a stroke.

Strokes. A common type of acquired brain injury resulting from endogenous causes treatable according to the invention is a stroke. A stroke is commonly understood to be a condition that adversely affects the blood supply to the brain. In one embodiment, the stroke is an ischemic stroke. Ischemic strokes include cardioembolic, atherosclerotic (thrombotic), embolic, thromboembolic, and cervical artery dissection. Ischemic strokes also include transient ischemic attacks (TIA) or mini-strokes. Thrombotic strokes are frequently caused by plaque buildup in major arteries supplying the brain.

In another embodiment, the stroke is a hemorrhagic stroke. A hemorrhagic stroke occurs when a weakened blood vessel ruptures. Two types of weakened blood vessels usually cause hemorrhagic stroke: aneurysms and arteriovenous malformations (AVMs). Aneurysms are weaknesses in blood vessel walls that bulge and can rupture. AVMs are abnormal, snarled tangles of blood vessels that cause multiple irregular connections between the arteries and veins. Types of hemorrhagic strokes include subarachnoid hemorrhage (SAH), which is bleeding in the space surrounding the brain, and intracerebral within-the-brain strokes. Intracranial hemorrhages include epidural hematoma, subdural hematoma, subarachnoid hemorrhage (SAH), intraventricular hemorrhage (IVH), hemorrhagic transformation of ischemic stroke (HT), venous hemorrhage from cortical vein or sinus thrombosis, and intracerebral hemorrhage (ICH).

In some embodiments, fasudil is used to treat patients who have had large vessel or small vessel strokes. Large vessel strokes affect one of the main arteries of the brain and form when blood clots from other parts of the body, such as the heart travel, to the brain. Large vessel strokes include those affecting the middle cerebral artery (MCA), the internal carotid artery (ICA), the posterior Inferior cerebellar artery (PICA), and the superior cerebellar artery (SCA). Small vessel strokes affect small branches of three large arteries—the middle cerebral artery (MCA), anterior cerebral artery, and posterior cerebral artery (PICA).

In one embodiment, the chronic stroke patient treated has a watershed stroke. A watershed stroke is an ischemic lesion which is situated along the border zones between the territories of two major arteries, for example the anteri- or and middle or the middle and posterior cerebral arteries. In a specific embodiment, the watershed stroke followed cardiac surgery.

In another embodiment, the chronic stroke patient treated with fasudil has an ischemic or hemorrhagic stroke in the cerebrum, cerebellum, sub-cortex or white matter, and brain stem.

Cerebral strokes. In one embodiment, fasudil is used to treat patients following cerebral strokes, including in the right and left hemisphere. About two thirds of strokes occur in the cerebrum. The effects of a right hemisphere cerebral stroke including left-sided weakness or paralysis and sensory impairment, visual problems, spatial problems with depth perception or directions, inability to localize or recognize body parts, inability to find objects, memory problems, and behavioral changes such as impulsivity, inappropriateness, depression.

The effects of a left hemisphere cerebral stroke include right sided weakness or paralysis and sensory impairment, problems with speech and understanding language (aphasia), visual problems, impaired ability to do math or to organize reason, and analyze items, behavioral changes such as depression, cautiousness, and hesitancy, impaired ability to read, write and learn new information, memory problems, and bowel and bladder control.

In some embodiments, fasudil is used to treat a cerebral stroke affecting the cerebral cortex. The cortex is the top layer of the cerebrum and is gray matter. Cortical strokes can occur in the frontal lobe, parietal lobe, occipital lobe, and temporal lobe.

In a specific embodiment, the stroke is a frontal lobe stroke. Frontal lobe strokes affect movement (muscle weakness) and language (aphasia, dysarthria, or apraxia), but also result in behavior, executive functioning, and memory problems and can cause seizures. In another specific embodiment, the stroke is a parietal lobe stroke. Parietal lobe strokes can cause a loss of sensation affecting one side of the face or body and affect language skills and problems reading and writing.

In another specific embodiment, the stroke is a temporal lobe stroke. Temporal lobe strokes mainly affect language and are likely to produce aphasia, specifically Wernicke's aphasia, if it occurs on the dominant side of the brain. In Wernicke's aphasia, the patient speaks but the speech lacks meaning. The patient has difficulty understanding written and spoken language. Motor deficits are typically not present. Hearing and vision may also be affected.

In another specific embodiment, the stroke is an occipital lobe stroke. This type of stroke is rare, but affects vision including central vision loss, cortical blindness, and visual hallucinations, because the brain's ability to process what the eyes are seeing is impaired.

In another specific embodiment, the stroke is a brain stem stroke. Brain stem strokes are characterized by vertigo and nausea and are difficult to diagnose. The strokes can be in the midbrain, pons, and medulla. The brain stem controls consciousness, breathing, swallowing, and other critical functions. Secondary effects of brain stem strokes include coma, whole body paralysis but for the eyes (locked in syndrome), difficulty breathing, dysphagia, nystagmus, ataxia, and loss of smell and taste.

Sub-cortical strokes. In another embodiment, fasudil is used to treat subcortical strokes. Sub cortical strokes affect the small vessels of the white matter below the cortex. In some embodiment, the small vessel strokes affect small branches of three large arteries—the middle cerebral artery (MCA), anterior cerebral artery, and posterior cerebral artery (PICA).

In some embodiments, small vessel strokes occur in the thalamus, basal ganglia, internal capsule, and brainstem.

In one embodiment, the stroke is in the thalamus. The thalamus controls sensory input. The right side of the thalamus transmits sensation from the left side of the body, and the left side of the thalamus transmits sensation from the right side of the body. A small vessel thalamic stroke can cause numbness, tingling, or even complete loss of sensation of the face, arm, and leg.

The basal ganglia controls sophisticated functions that require coordination and muscle movements. A small vessel stroke affecting the basal ganglia can cause symptoms such as writhing, twisting movements, muscle twitching, and/or tremor.

The internal capsule is a deep brain, myelinated structure that transmits signals for movement to more inferior structures. Symptoms of a small vessel stroke in this area may include mild weakness, severe weakness, numbness, or complete paralysis of the opposite face, arm, and leg.

In a further embodiment, the stroke is a lacunar stroke. Lacunar strokes, a type of ischemic stroke, are small and located in non-cortical areas. The location and accumulation of multiple lacunar infarctions can lead to significant physical and cognitive disabilities. persistent or transient numbness and/or tingling on one side of the body.

In another embodiment, the patient to be treated with fasudil has CADASIL (Cerebral Autosomal Dominant Arteriopathy with Sub-cortical Infarcts and Leukoencephalopathy) is an inherited form of cerebrovascular disease that occurs when the thickening of blood vessel walls blocks the flow of blood to the brain. By age 65, the majority of persons with CADASIL have cognitive problems and dementia.

Cerebellar strokes. In another embodiment, fasudil is used to treat cerebellar strokes. Common effects of cerebellar strokes are inability to walk and problems with coordination and balance (ataxia), dizziness, headache, nausea, and vomiting.

The different types and locations of strokes are diagnosed by clinicians with the aid of neuroimaging.

In one embodiment, the chronic stroke patient treated with fasudil has hypertension.

In another embodiment, the chronic stroke patient treated with fasudil does not have hypertension.

In one embodiment, the chronic stroke patient treated with fasudil has Type II diabetes. In one embodiment, the chronic stroke patient treated with fasudil does not have Type II diabetes.

In one embodiment, the chronic stroke patient treated with fasudil has high cholesterol.

In another embodiment, the chronic stroke patient treated with fasudil does not have high cholesterol.

In one embodiment, the chronic stroke patient treated with fasudil is overweight or obese. In another embodiment, the chronic stroke patient treated with fasudil is not overweight or obese.

In one embodiment, the chronic stroke patient treated with fasudil is a smoker. In another embodiment, the chronic stroke patient treated with fasudil is not a smoker. In one embodiment, the chronic stroke patient treated with fasudil has one or more of coronary artery disease, atrial fibrillation, or heart valve disease.

In another embodiment, the chronic stroke patient treated with fasudil does not have one or more of coronary artery disease, atrial fibrillation, or heart valve disease. In one embodiment, the chronic stroke patient treated with fasudil has an elevated hematocrit. In a specific embodiment, the patient has polycythemia.

In one embodiment, the chronic stroke patient treated with fasudil does not exhibit disorientation or wandering, including getting lost in familiar places.

In another embodiment, the chronic stroke patient treated with fasudil does not have sub-cortical vascular dementia, such as Binswanger's disease.

In another embodiment, the chronic stroke patient treated did not suffer from a lacunar stroke.

In a further embodiment, the chronic stroke patient treated with fasudil does not have dementia or mild cognitive impairment (MCI). In a specific embodiment, the patient has a score on the Mini-Mental State Examination (MMSE) of about 24 to 30.

In another embodiment, the chronic stroke patient treated with fasudil has mild dementia. In one embodiment, the patient has an MMSE score of 18 to 23.

In one embodiment, the chronic stroke patient treated with fasudil has moderate dementia. In a specific embodiment, the patient has an MMSE score of about 13 to 20.

In one embodiment, the chronic stroke patient treated with fasudil, has amyotrophic lateral sclerosis (ALS). In a specific embodiment, the ALS patient treated previously had a cerebral AVM or TIA.

In another embodiment, the patient does not have ALS.

In one embodiment, the chronic stroke patient treated with fasudil is African American or Hispanic.

In another embodiment, the chronic stroke patient treated with fasudil is female. In a specific embodiment, the patient has chronically used birth control pills.

TBI. A common type of acquired brain injury resulting from exogenous causes treatable according to the invention is a traumatic brain injury (TBI). TBI refers to damage to the brain resulting from external mechanical force. The Center for Disease Control (CDC) defines TBI as a head injury resulting from blunt or penetrating head trauma or from acceleration/deceleration forces resulting in any of the following: altered level of consciousness, loss of memory, neurologic abnormality, skull fracture, diagnosed intracranial lesions, or head injury listed as a cause of death in the death certificate (Thurman, Sniezek, Johnson, Greenspan, & Smith, 1995). While the extent of TBI can vary significantly, TBI is a complex injury, consisting of a primary insult (i.e., primary damage, mechanical damage) occurring at the moment of impact that causes damage to brain structures, together with the secondary insult (secondary damage, delayed non-mechanical damage), which represent consecutive pathological processes initiated at the moment of injury with delayed clinical presentation.

In one embodiment, TBIs, irrespective of cause or type may be treatable according to the invention.

In an embodiment, the subject has acquired TBI while participating in a contact sport.

In another embodiment, the subject has acquired TBI through an accident or a fall.

In other embodiments, TBIs can result from falls, firearm wounds, sports accidents, construction accidents and vehicle accidents, among other causes. Blast injuries are common among military service members and veterans.

In yet other embodiments, TBIs can occur from lacerations, skull fractures, and conversely, even in the absence of external signs of head injury. A traumatic brain injury may result from a blow to the head and manifest as either an open or closed injury and the disclosure contemplates treatment of blunt TBIs and penetrating TBIs. The blow to the head leads to rapid brain tissue displacement, which may result in disruption of vascular channels, hemorrhage (including contusion), direct tissue injury and edema. TBIs can result in parenchymal (brain tissue) injury and or vascular (blood vessel) injury. As a type of TBI, for example, a concussion typically results in parenchymal injury with underlying diffuse axonal injury that results from stretching and distortion of the axons, which may result in axonal retraction and, ultimately, neuronal cell death.

TBI is classified according to its severity: mild, moderate or severe. Classification may be clinically determined based on the Glasgow Coma Scale (GCS), which assesses motor, verbal and eye-opening responses. A subject with mild TBI will have a GCS of between 13 and 15; a subject with moderate TBI will have a GCS of between 9 and 12; and a subject with severe TBI will have a GCS less than 9. In the absence of a clinical assessment, TBI is considered moderate to severe if there is a loss of consciousness that is longer than 30 minutes and amnesia that lasts for more than 24 hours. If these conditions are not met, TBI is classified as mild. Concussion in the absence of other symptoms is typically classified as a mild TBI. TBI patients can suffer from a number of physical, cognitive, social, emotional and/or behavioral disorders following injury, which may be long-lasting or even permanent.

In an embodiment, the TBI is mild to severe TBI. In another embodiment, the TBI is moderate to severe TBI.

TBI has chronic effects and long-term consequences and there are few therapeutic interventions for such chronic effects. Long-term consequences of TBI include seizures/epilepsy, including subclinical, non-convulsive seizures, sleep disorders, neurodegenerative diseases, neuroendocrine dysregulation, psychiatric problems demyelination, gray and white matter atrophy, and decreased neurogenesis.

TBI survivors can experience a wide range of deficits, and sensorimotor as well as cognitive impairment is a common consequence of this injury. Sensorimotor impairment includes elements of paresis, postural imbalance and gait disturbance, and early acute disruption of the startle reflexes. TBI can result in bradykinesia, abnormal sway, and an impaired reaction time. Early balance impairment is a predictor of worse outcome post-TBI. Sensorimotor problems may improve over time, although depending on severity deficits may persist beyond the first 1-2 years after trauma.

In cognitive domains, impairments are observed in memory, attention and information processing speed, with more severe TBI causing greater and longer-lasting deficits than mild or moderate TBI. TBI survivors frequently present with increased anxiety, agitation and disinhibition in behavior.

According to the diagnostic criteria detailed in the “Diagnostic and Statistical Manual of Mental Disorders (DSM-5) TBI has one or more of the following characteristics: changes in levels of consciousness; memory disturbances; confusion associated with deficits in orientation; neurological signs, such as brain injury observable on neuroimaging, new onset or worsening of seizure disorder, visual field deficits and hemiparesis.

### Pharmaceutical Compositions

A pharmaceutical composition comprising a ROCK inhibitor such as fasudil and/or a salt thereof may comprise one or more pharmaceutically acceptable excipients, which are known in the art. Formulations of fasudil are generally oral and include oral films, orally disintegrating tablets, effervescent tablets and granules or beads that can be sprinkled on food or mixed with liquid as a slurry or poured directly into the mouth to be washed down. Liquid oral dosage forms are also contemplated.

Pharmaceutical compositions containing ROCK inhibitors, salts and hydrates thereof can be prepared by any method known in the art of pharmaceutics. In general, such preparatory methods include the steps of bringing a ROCK inhibitor or a pharmaceutically acceptable salt thereof into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. The composition used in accordance with the methods of the present invention may comprise between 0.001% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

In certain embodiments, the pharmaceutical composition used in the methods of the present invention may comprise a diluent. Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

In certain embodiments, the pharmaceutical composition used in the methods of the present invention may comprise a granulating and/or dispersing agent. Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM), sodium lauryl sulfate, quaternary ammonium compounds, and mixtures thereof.

In certain embodiments, the pharmaceutical composition used in the methods of the present invention may comprise a binding agent. Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan), alginates, polyethylene oxide, polyethylene glycol, inorganic calcium salts, silicic acid, polymethacrylates, waxes, water, alcohol, and/or mixtures thereof.

In certain embodiments, the pharmaceutical composition used in the methods of the present invention may comprise a preservative. Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

In certain embodiments, the pharmaceutical composition used in the methods of the present invention may comprise an antioxidant. Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

In certain embodiments, the pharmaceutical composition used in the methods of the present invention may comprise a chelating agent. Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

In certain embodiments, the pharmaceutical composition may comprise a buffering agent together with the ROCK inhibitor or the salt thereof. Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

In certain embodiments, the pharmaceutical composition used in the methods of the present invention may comprise a lubricating agent. Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

In other embodiments, the pharmaceutical composition containing a ROCK inhibitor such as fasudil or salt thereof will be administered as a liquid dosage form. Liquid dosage forms for oral and parenteral administration include pharmaceutically acceptable emulsions, microemulsions, solutions, suspensions, syrups, and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates of the invention are mixed with solubilizing agents such as Cremophor™, alcohols, oils, modified oils, glycols, polysorbates, cyclodextrins, polymers, and mixtures thereof. Liquid dosage forms also include oral and parenteral liposomes.

In other embodiments, the pharmaceutical composition of containing a ROCK inhibitor such as fasudil or salt thereof will be administered as a solid dosage form. Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Some compositions of ROCK inhibitors such as fasudil relate to extended- or controlled-release formulations. These may be, for example, diffusion-controlled products, dissolution-controlled products, erosion products, osmotic pump systems or ionic resin systems. Diffusion-controlled products comprise a water-insoluble polymer which controls the flow of water and the subsequent egress of dissolved drug from the dosage from. Dissolution-controlled products control the rate of dissolution of the drug by using a polymer that slowly solubilizes or by microencapsulation of the drug-using varying thicknesses to control release. Erosion products control release of drug by the erosion rate of a carrier matrix. Osmotic pump systems release a drug based on the constant inflow of water across a semi permeable membrane into a reservoir which contains an osmotic agent. Ion exchange resins can be used to bind drugs such that, when ingested, the release of drug is determined by the ionic environment within the gastrointestinal tract.

### Dosage and Administration

In accordance with the treatment methods disclosed herein, an effective amount of a ROCK inhibitor such as fasudil or a pharmaceutically acceptable salt thereof for administration one or more times a day begins more than three months following the stroke.

Fasudil hydrochloride hemihydrate, for example, is suitably administered in a daily amount exceeding 60 mg/day. In one embodiment, fasudil is administered in a dose of about 65 mg to about 500 mg, about 65 mg to about 400 mg, about 65 mg to about 200 mg, about 65 mg to about 100 mg. One exemplary dosing regimen involves the treatment with Fasudil hydrochloride hemihydrate three times per day using an immediate-release formulation, for a total daily dose of 70-240 mg. Another exemplary dosing regimen is administering 70 mg to 180 mg per patient per day administered in three equal amounts during the day. Still another exemplary daily dose is 90 mg per patient per day, administered in two doses of 45 mg twice a day in immediate release formulation. A further dosing regimen involves the treatment with 35 to 9 mg of Fasudil hydrochloride hemihydrate two times per day using an immediate-release formulation, for a total daily dose of 70-180 mg.

Certain patient sub-populations, such as renally impaired patients and/or older patients (e.g., 65 or older) may need lower doses or extended release formulations instead of immediate release formulations. Fasudil hydrochloride hemihydrate may have higher steady-state concentrations when given at usual doses to patients with renal disease and lower doses to lower the Cmax or delay the time to Cmax (increase the Tmax) may be required.

In addition, older patients may need a lower dose at initiation, with a gradual increase to the recommended dose after days or weeks. In another embodiment, older patients may need lower doses for the duration of treatment. The aged population includes the “young old” who are 65-74, the “old old” who are 75-84 and the “frail elderly” who are 85 and older. For example, a starting dose of 30 mg per day for two weeks, followed by 60 mg per day for 4 weeks, then by 90 mg per day. Titration may even be warranted up to about 120 mg per day.

Another embodiment involves the treatment with 60-120 mg of Fasudil hydrochloride hemihydrate once per day in an extended-release dosage form. Treatment with an extended-release total daily dose of 90 mg Fasudil hydrochloride hemihydrate is preferred.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

In one embodiment, administration of the ROCK inhibitors, such as fasudil, to a chronic stroke patient begins more than three months following a stroke.

In another embodiment, administration begins about four months following a stroke.

In a further embodiment, administration begins about five months following a stroke.

In other embodiments, administration begins about six months, seven months, eight months, nine months, ten months, eleven months or twelve months following a stroke.

In a specific embodiment administration begins more than three months to six months following the stroke.

Administration would generally be continued for at least one week. In some embodiments daily administration continues for up to 30 days or up to 60 days or up to 90 days or even more. Treatment for more than 60 days is preferred and treatment for at least six months is particularly preferred. The precise duration of treatment will depend on the patient's condition and response to treatment.

### Assessing Improvements in Deficits Upon Fasudil Treatment.

Improvements upon fasudil treatment can be evaluated using numerous assessments accepted in the field. In one specific embodiment, the deficits disclosed above can be measured at baseline, right before treatment, and then during and/or following treatment with the ROCK inhibitor, using assessments accepted in the art. The change from baseline represents the improvement.

In one embodiment, treatment of any of the aforementioned chronic stroke patients or TBI with fasudil improves the deficits associated with stroke or TBI patients respectively. The skilled person will understand that some assessments can be used for both stroke or TBI although some are specific for one or the other.

In a specific embodiment, efficacy of fasudil treatment is measured by assessment of daily living activities. In one embodiment, treatment is assessed using the modified Rankin Score (mRS). The mRS is a disability scale commonly used to assess stroke patients. The scale runs from 0-6, running from perfect health without symptoms to death.

| SCORE | DESCRIPTION |
|---|---|
| 0 | No symptoms. |
| 1 | No significant disability. Able to carry out all usual |
| | activities, despite some symptoms. |
| 2 | Slight disability. Able to look after own affairs without |
| | assistance, but unable to carry out all previous activities. |
| 3 | Moderate disability. Requires some help, but able to walk |
| | unassisted. |
| 4 | Moderately severe disability. Unable to attend to own bodily |
| | needs without assistance, and unable to walk unassisted. |
| 5 | Severe disability. Requires constant nursing care and |
| | attention, bedridden, incontinent. |
| 6 | Dead. |

In an embodiment, fasudil treatment beginning more than three months following stroke results in an improvement in the score from baseline (excluding dead).

Similarly, in assessing TBI, the Glasgow Outcome Scale is often employed. It may be administered as a 5-point scale (the GOS), collapsing upper and lower categories, or as an extended 8-point scale (the GOSE)

| Score | GOSE | GOS |
|---|---|---|
| 1 | Death | Death |
| 2 | Vegetative state | Vegetative state |
| 3 | Lower severe disability | Severe disability |
| 4 | Upper severe disability | Moderate disability |
| 5 | Lower moderate disability | Good recovery |
| 6 | Upper moderate disability | — |
| 7 | Lower good recovery | — |
| 8 | Upper good recovery | — |

A structured interview is often employed to ensure consistent scoring (Wilson 1997).

In an embodiment, fasudil treatment beginning more than three months following TBI results in an improvement in the score from baseline (excluding dead).

In another embodiment, efficacy of brain injury patients is assessed using the Barthel Index (BI). Barthel Index (BI) is a measure of activities of daily living (ADL). It assesses 10 different domains, each on a 5-15 point scale, resulting in possible scores ranging from 0 (completely dependent) to 100 (completely independent).

Generally, a score of 80-100 indicates the patient is able to live independently, 60-79 indicates minimal dependence and requiring occasional assistance in daily live, 40-59 indicates partial dependence and requiring regular assistance with some activities, 20-39 very dependent and requiring regular assistance with a number of activities, and <20 indicates complete dependence and the inability to function without assistance.

| DOMAIN | SCORING |
|---|---|
| FEEDING | 0 = unable |
| | 5 = needs help cutting, spreading butter, etc., |
| | or requires modified diet |
| | 10 = independent |
| BATHING | 0 = dependent |
| | 5 = independent (or in shower) |
| GROOMING | 0 = needs to help with personal care |
| | 5 = independent face/hair/teeth/shaving |
| | (implements provided) |
| DRESSING | 0 = dependent |
| | 5 = needs help but can do about half unaided |
| | 10 = independent (including buttons, zips, |
| | laces, etc.) |
| BOWELS | 0 = incontinent (or needs to be given enemas) |
| | 5 = occasional accident |
| | 10 = continent |
| BLADDER | 0 = incontinent, or catheterized and unable |
| | to manage alone |
| | 5 = occasional accident |
| | 10 = continent |
| TOILET USE | 0 = dependent |
| | 5 = needs some help, but can do something alone |
| | 10 = independent (on and off, dressing, wiping) |
| TRANSFERS | 0 = unable, no sitting balance |
| (BED TO | 5 = major help (one or two people, physical), can sit |
| CHAIR | 10 = minor help (verbal or physical) |
| AND BACK) | 15 = independent |
| MOBILITY | 0 = immobile or <50 yards |
| (ON LEVEL | 5 = wheelchair independent, including |
| SURFACES) | corners, >50 yards |
| | 10 = walks with help of one person (verbal or |
| | physical) >50 yards |
| | 15 = independent (but may use any aid; for |
| | example, stick) >50 yards |
| STAIRS | 0 = unable |
| | 5 = needs help (verbal, physical, carrying aid) |
| | 10 = independent |

### The Barthel ADL Index: Guidelines

1. The index should be used as a record of what a patient does, not as a record of what a patient could do.
2. The main aim is to establish a degree of independence from any help, physical or verbal, however minor and for whatever reason.
3. The need for supervision renders the patient not independent.
4. A patient's performance should be established using the best available evidence. Asking the patient, friends/relatives and nurses are the usual sources, but direct observation and common sense are also important. However direct testing is not needed.
5. Usually the patient's performance over the preceding 24-48 hours is important, but occasionally longer periods will be relevant.
6. Middle categories imply that the patient supplies over 50 percent of the effort.
7. Use of aids to be independent is allowed.

In an embodiment, fasudil treatment beginning more than three months following brain injury results in an improvement in the BI score from baseline.

Cognitive deficit assessment. In yet another embodiment, cognitive deficits are assessed in brain injury patients. In one embodiment, the change from baseline prior to treatment with fasudil is assessed using accepted cognitive assessments. One of skill in the art can select the most appropriate assessment, based on the patient's pre-injury cognitive functioning and the type and severity of the injury incurred.

In one embodiment, baseline cognitive deficits are assessed using the MMSE (Folstein test). The maximum MMSE score is 30 points. A score of 20 to 24 suggests mild dementia, 13 to 20 suggests moderate dementia, and less than 12 indicates severe dementia.

In another embodiment, baseline cognitive deficits are assessed using the Montreal Cognitive Assessment (MoCA). MoCA assesses short term memory, visuospatial abilities, executive functions, attention, concentration, and working memory, language, and orientation as to time and place. A MoCA score of 26 or higher is generally considered normal, while a score of 18 to 25 can indicate mild cognitive impairment, and 10 to 17 can indicate moderate impairment. A score of less than 10 indicates severe impairment.

The following table presents a list of common cognitive tests with which one skilled in the art will be familiar and which are helpful in assessing the outcomes of the disclosed methods of treatment.

### Common Cognitive Tests

| | |
|---|---|
| Test | Description |

Processing Speed

| | |
|---|---|
| Pattern | Determine whether a set of two pictures placed side |
| Comparison | by side are the same for 90 seconds. |
| Processing | |
| Speed Test | |
| Digit Symbol | Timed measure consisting of nine digit-symbol pairs; |
| Substitution | participant matches the correct symbol to a list of |
| Test | digits. |

Sustained attention

| | |
|---|---|
| Connors | Measure selective response to target inputs while |
| Continuous | doing a repetitive task -- for example, respond only |
| Performance | when they will see or hear the number 1, but not the |
| Test | number 2. |

Selective attention

| | |
|---|---|
| Target Letter | Search a visual display for a target, such as a letter, |
| Search | that is surrounded by other, non-target letters. |
| Stroop Test | Name the color of ink in which another color word is |
| | printed (e.g., the word “red” printed in the color |
| | green). |

Episodic memory

| | |
|---|---|
| Picture Sequence | Recall series of 6 to 18 illustrated objects and |
| Memory Test | activities presented in a certain order. |

Working memory

| | |
|---|---|
| General | Common tests include giving participants a list of |
| | items (letters, numbers, words) and asking them to |
| | repeat the items back in order. |
| List Sorting | Participants see pictures and written names of foods |
| Test | and animals and hear audio names. The participant |
| | recites them back in order of size from smallest to |
| | largest, first within a single category (e.g., animals) |
| | and then in two categories (e.g., foods, then animals). |

Semantic memory

| | |
|---|---|
| Category | Name all of the items they can in a category (e.g., |
| Fluency Test | animals) in 1 minute. |
| Boston | Subject sees a series of pictures and must name each |
| Naming Test | picture within 20 seconds |

Executive function

| | |
|---|---|
| Wisconsin Card | Subject matches two sets of cards according to some |
| Sorting Test | characteristic, but the participant is not told the rules - |
| | they learn through feedback. After a fixed number |
| | of correct matches, the rules are changed without |
| | notice, and the new rule must be learned. |
| Trail | Subject connects 25 consecutive numbers or |
| Making Test | alternating numbers and letters in ascending order. |

Reasoning

| | |
|---|---|
| Letter | consists of five sets of four letters. The participant has |
| Sets Test | to decide which one of the five sets is dissimilar in the |
| | sense that it does not follow a rule used to generate |
| | the other four sets. Tests of inference are used to |
| | measure the ability to reason and draw conclusions |
| | from information presented in statements. |

Language

| | |
|---|---|
| General | There are a variety of tests, depending on the |
| | particular aspect of language being assessed. These |
| | include a word-by-word reading paradigm to assess |
| | comprehension of sentences (e.g., Kemtes and |
| | Kemper, 1997), word-by-word reading time (e.g., |
| | Stine, 1990) or recall of text (Stine-Morrow et al., |
| | 1996). |
| Token Test | Subject presented with tokens that vary in shape color |
| | and size and must follow commands related to |
| | identifying them (e.g., touch the circle). |
| Boston | Confrontational naming test - subject shown objects |
| Naming Test | to name in certain period of time. |
| Category | Subject is asked to name items in a category, like |
| Fluency Test | words beginning with F, A and S, or as many animals |
| | as possible in a particular time. |
| Boston | A testing battery that evaluates language skills based |
| Diagnostic | on perceptual modalities (auditory, visual, and |
| Aphasia | gestural), processing functions (comprehension, |
| Examination | analysis, problem-solving), and response modalities |
| | (writing, articulation, and manipulation). |

Spatial ability

| | |
|---|---|
| Paper | Decide which pieces of paper with holes punched in |
| Folding Test | them is the same by looking at them folded and |
| | unfolded. |
| Mental | Compare several three-dimensional objects that are |
| Rotations | rotated differently and state whether they are the |
| Test | same or mirror images. |
| Block | Subtest of the Wechsler Adult Intelligence Scale |
| Design Test | (WAIS). Arrange a set of 3-dimensional blocks to |
| | match a 2-dimensional drawing using color cues |
| | within a specific time limit. |

Intelligence

| | |
|---|---|
| Wechsler | Consists of 10 core subtests and 5 supplemental |
| Adult | subtests. The core subtests provide the intelligence |
| Intelligence | quotient, which is derived from four index scores: |
| Scale | Verbal Comprehension Index, Perceptual Reasoning |
| | Index, Working Memory Index, and Processing Speed |
| | Index. |

Motor deficits assessment. Motor deficits following brain injuries, including TBI and stroke include tremor, impaired balance, increased reaction time, and reduced visuomotor coordination. In addition to the Glasgow Coma Scale, motor deficits can also be measured as a change from baseline using accepted assessments such as the StrokEDGE and StrokEDGE II Scoring Matrix (Sullivan 2013).

In another embodiment, motor deficits can be assessed using the National Institutes of Health Stroke Scale (NIHSS), which measures both initial assessments and ongoing severity. (Hinkle 2014). NIHSS measures 15 clinical functions or deficits such as gaze, vision, level of consciousness and facial palsy. Limb ataxia, sensory loss, dysarthria and language are also measured. Arm and leg strength, along with inattention, are also evaluated. The items must be administered in order and without patient coaching.

Other motor assessment tests include the Berg Balance Scale (Berg 1989). The assessment requires an individual to complete instructed tasks (such as unsupported standing, looking behind, and standing on one foot) while maintaining balance. The Community Balance and Mobility Scale is designed to measure stability, balance, postural control, mobility, and walking ability (Howe et al., 2006). Each of the 13 tasks involved in the assessment are graded on a nominal scale from 0 to 5, for a possible total score of 96. Individuals must have some level of ambulation in order to participate in this assessment. The function of this assessment is to determine whether or not an individual has the mobility and functionality necessary to participate in the community. Comparisons are performed using a scale of healthy individuals stratified by age groups.

The Modified Ashworth Scale (MAS) is a clinician administered assessment of spasticity and tone. The MAS measures the resistance during passive stretching in eight areas of the body from the fingers to the soleus. Each area is measured on a nominal scale from 0-4, with 0 representing normal tone and 4 representing extreme rigidity, and is conducted in the supine position.

In a further embodiment, prediction of motor recovery can be assessed using MRI or diffusion tensor imaging. DTI is a MRI sequence that specifically evaluates the directionality and integrity of brain axonal fibers and network strength in case of neuronal injury due to various etiologies including stroke and TBI. DTI studies have established fractional anisotropy (FA) values as surrogate markers of motor deficit after stroke: worse motor function usually relates to lower values in affected corticospinal tract. (Darwish 2021).

In another embodiment, motion capture systems (MCS) have been used to assess motor function in different neurological conditions. The most commonly used system is Microsoft Kinect®, which is a portable and marker-free motion capture system that uses an infrared light and a deep sensor to create a three-dimensional reconstruction of the human body and detect its movements. (Venugopalan 2013) Kinect® results are concordant with marker-based systems. Studies have evaluated the feasibility of this system for gait assessment in multiple sclerosis or Parkinson disease and for upper extremity motor function evaluation in muscle diseases.

In another embodiment, assessment can be done using the Fugl-Meyer Assessment (FMA). (Fugl-Meyer 1975). The FMA is a stroke-specific, performance-based impairment index. It is designed to assess motor functioning, balance, sensation and joint functioning in patients with post-stroke hemiplegia. The scale is comprised of five domains and there are 155 items in total: Motor functioning (in the upper and lower extremities); Sensory functioning (evaluates light touch on two surfaces of the arm and leg, and position sense for 8 joints); Balance (contains 7 tests, 3 seated and 4 standing); Joint range of motion (8 joints); and Joint pain. Scoring is based on direct observation of performance. Scale items are scored on the basis of ability to complete the item using a 3-point ordinal scale where 0=cannot perform; 1=performs partially; and 2=performs fully. The total possible scale score is 226.

Motor score: ranges from 0 (hemiplegia) to 100 points (normal motor performance). Divided into 66 points for upper extremity and 34 points for the lower extremity.

Sensation: ranges from 0 to 24 points. Divided into 8 points for light touch and 16 points for position sense.

Balance: ranges from 0 to 14 points. Divided into 6 points for sitting and 8 points for standing.

Joint range of motion: ranges from 0 to 44 points.

Joint pain: ranges from 0 to 44 points.

The proposed classifications of impairment severity are: 0-35=very severe; 36-55=severe; 56-79=moderate; and greater than 79=mild. (Duncan 1994).

In another embodiment, motor assessments are done using the Trunk Impairment Scale (TIS). The TIS measures the motor impairment of the trunk after a stroke through the evaluation of statis and dynamic sitting balance, as well as coordination of trunk movement. (Verheyden 2004; Verheyden 2010). Each subscale has between three and ten items. Scores range from a minimum of zero to a maximum of 23.

In another embodiment, motor ability is assessed using the Functional Ambulation Categories. The Functional Ambulation Categories (FAC) is a 6-point functional walking test that evaluates ambulation ability, determining how much human support the patient requires when walking, regardless of whether or not they use a personal assistive device. Only stairs and 15 meters of indoor floor are needed to administer the test. (Mehrholz 2007). The rating scale is from 0 to 5 where zero is a non-functional ambulator; 1=a patient who requires continuous manual contact to support body weight and to maintain balance or assist coordination; 2=a patient who requires intermittent of continuous light touch to assist balance or coordination; 3=a patient who can walk on a level surface without manual contact but requires standby of another for safety or verbal cueing; 4=a patient who can ambulate independently on level surfaces but requires supervision to negotiate stairs, inclines or non-level surfaces; 5=a patient who can walk everywhere independently.

There is also a Modified Functional Ambulation Classification. (Chau M 2013). The MFAC is a 7-point Likert Scale (I to VII) that is used to classify a patient's walking capacity. Gait is divided into seven categories, ranging from no ability to walk and requires manual assistance to sit or is unable to sit for 1 minute without back or hand support (MFAC I) to the ability to walk independently on level and non-level surfaces, stairs, and inclines (MFAC VII)

In another embodiment the Functional Independence Measurement (FIM) is used to assess effects following treatment. The FIM (Guide for the Uniform Data Set for Medical Rehabilitation, 1996) is a widely accepted functional assessment measure in use in the rehabilitation community. The FIM is an 18-item ordinal scale, used with all diagnoses within a rehabilitation population. It is viewed as most useful for assessment of progress during inpatient rehabilitation. (Fielder 1996; Graham 2015)

In other embodiments, psychosocial assessments used include General Health Questionnaire (GHQ), Quality of life (SAQOL and Stroke Specific QoL), sense of coherence (SOC), and depression (Yale). The Stroke Specific QOL (Williams L S 1999) assesses behavior; cognition; functional mobility; language; negative affect; personality; quality of life; social relationships; and upper mobility function. There are 49 items that are assessed on 5-point Guttman-type scale. Scores range from 49 to 245 with the higher score indicating better functioning.

The Sense of Coherence Scale (SOC) assesses how people view life and identifies how they use their resistance resources to maintain and develop their health. (Antonovsky 1993). The Sense of Coherence Scale has 29 items with response options for each item using 7-point Likert scale: (e.g., with the example above: 1, “No clear goal or purpose at all” to 7, “Very clear goals and purpose”). Items are summed, yielding a range from 7 to 203. Higher scores indicate greater levels of sense of coherence.

Depression scales such as the Beck Depression Inventory and the Yale Depression scale also can be used to measure psychosocial deficits. The Beck Depression Inventory (BDI) is a 21-item, self-report rating inventory that measures characteristic attitudes and symptoms of depression (Beck 1961). There is also a the 13-item short form and the more recent BDI-II by Beck, Steer & Brown, 1996. (Steer 2000). The Yale Depression Scale is a 25-question scale that was designed for predicting treatment responses. (Chekroud 2016)

Use of a combination of the foregoing assessments is also contemplated.

Neurobehavioral Deficits. Many general assessment tools are available to assess neurobehavioral deficits and these include the Patient Health Questionnaire-9 (PHQ-9), Neurobehavioral Core Test Batter (NCTB), Neurobehavioral Evaluation System (NES), Neuropsychiatric inventory questionnaire (Depression and Neuropsychiatric conditions). If a certain domain is identified and more sensitivity is desired, more specific scales known to one of skill in the art include the Geriatric Depression Scale: Short Form (Depression), Beck's depression inventory (Depression), Columbia suicide severity score (Suicidality), Hamilton depression scale (Depression), Hospital anxiety and depression scale (Anxiety and depression), the Cornell Scale for Depression in Dementia (Depression) and the Dementia Mood Assessment Scale (DMAS; Depression), and the Cohen-Mansfield Agitation Inventory (CMAI; Agitation symptoms). Another potential biomarker is α-spectrin in CSF, which is a calpain-specific breakdown product. Prolonged activation of calpains is associated with TBI.

Biomarkers. Inflammation is a consequence of TBI and therefore biomarkers include the presence of activated inflammatory cells and the presence of increased chemokines and cytokines that can be proinflammatory and thus potentially neurotoxic. Biochemical analyses have indicated that increased elevations in tissue levels of interleukin (IL)-1β, tumor necrosis factor alpha (TNF-α), IL-6, and transforming growth factor as well as many others are observed at various times after TBI. C1q has also been found at high levels in the thalamus months after the initial injury in animal models. The high C1q levels were associated with neuronal death, inflammation.

### Combination Therapy

The ROCK inhibitors such as fasudil can be administered alone or in combination with rehabilitation including physical and occupational therapy. Combination therapy with pharmacological agents such as anti-hypertensives, beta-blockers, ACE inhibitors, anti-depressants, and diuretics, for stroke patients is also contemplated.

All references cited herein are incorporated by reference in their entireties.

### LIST OF REFERENCES

Antonovsky, A. The structure and properties of the Sense of Coherence Scale. Soc Sci Med. 1993; 36 (6), 725-733.
Beck, A. T., Ward, C. H., Mendelson, M., Mock, J., & Erbaugh, J. (1961) An inventory for measuring depression. Archives of General Psychiatry, 4, 561-571.
Chau M W Rosanna et al., Reliability and validity of the Modified Functional Ambulation Classification in patients with hip fracture. Hong Kong Physiotherapy Journal. 2013; 31:41-44.
Chen M, Liu A, Ouyang Y, Huang Y, Chao X, Pi R. 2013. Fasudil and its analogs: a new powerful weapon in the long war against central nervous system disorders? Expert Opin Investig Drugs. 22:537-50.
Cheng C I Lin Y C Tsai T H The prognostic values of leukocyte rho kinase activity in acute ischemic stroke. Biomed Res Int. 2014; 2014:214587.
Chekroud A M et al., “Cross-trial prediction of treatment outcome in depression: a machine learning approach. Lancet Psychiatry. 2016. 3 (3): 243-50.
Darwish H S et al., Prediction of Motor Recovery after Stoke by Assessment of Corticospinal Tract Wallerian Degeneration Using Diffusion Tensor Imaging. Indian J Radiol Imaging 2021; 31:131-37.
Duncan P W, Goldstein L B, Horner R D, Landsman P B, Samsa G P, Matchar D B. Similar motor recovery of upper and lower extremities after stroke. Stroke. 1994 Jun. 1; 25 (6): 1181-8.
Feng Y, LoGrasso P, Defert O, Li R, Rho Kinase (ROCK) Inhibitors and Their Therapeutic Potential. J Med Chem. 2016; 59*6): 2269-2300.
Fielder R C and Granger C V (1996). The Functional Independence Measure: A Measurement of Disability and Medical Rehabilitation. In: Chino, N., Melvin, J. L. (eds) Functional Evaluation of Stroke Patients. Springer, Tokyo.
Fugl-Meyer A R, Jääskö L, Leyman I, Olsson S, Steglind S. The post-stroke hemiplegic patient. 1. a method for evaluation of physical performance. Scandinavian journal of rehabilitation medicine. 1975; 7 (1): 13-31.
Graham J E et al., The Uniform Data System for Medical Rehabilitation. Am J Phys Med Rehabil. 2014 March; 93 (3): 231-244.
Hinkle J L. Reliability and validity of the National Institutes of Health stroke scale for neuroscience nurses. Stroke. 2014; 4 5 (3): e32-e34.
Kamei S, Oishi M, Takasu T. 1996a. Evaluation of fasudil hydrochloride treatment for wandering symptoms in cerebrovascular dementia with 31P-magnetic resonance spectroscopy
and Xe-computed tomography. Clin Neuropharmacol. 19:428-38.
Kamei S, Toshiaki T, Oishi M, Effect of fasudil hydrochloride on wandering symptoms of c cerebrovascular dementia patients. Neurotherapy. 1996b 13:43-50.
Lee, KkB et al., Six-month functional recovery of stroke patients a multi-time-point study. International Journal of Rehabilitation Research. 2015; 38 (2): 173-80.
Mehrholz, J., Wagner, K., Rutte, K., Meiner, D. and Pohl, M. Predictive validity and responsiveness of the Functional Ambulation Category in hemiparetic patients after stroke. Archives of Physical Medicine Rehabilitation, 2007, 88, 1314-1319.
Nishikimi T Koshikawa S Ishikawa Y, Inhibition of Rho-kinase attenuates nephrosclerosis and improves survival in salt-loaded spontaneously hypertensive stroke-prone rats. J Hypertens. 2007; 25:1053-1063.
Rikitake Y Kim H H Huang Z Inhibition of Rho kinase (ROCK) leads to increased cerebral blood flow and stroke protection. Stroke. 2005; 36:2251-2257.
Shibuya M, Asano T, Sasaki Y. 2001. Effect of Fasudil HCl, a protein kinase inhibitor, on cerebral vasospasm. Acta Neurochir Suppl. 77:201-4.
Shin H K Salomone S Ayata C. Targeting cerebrovascular Rho-kinase in stroke. Expert Opin Ther Targets. 2008; 12:1547-1564. 46.
Steer, R. A., Rissmiller, D. J.& Beck, A. T., (2000) Use of the Beck Depression Inventory with depressed geriatric patients. Behaviour Research and Therapy, 38 (3), 311-318.
Sullivan J. E. et al., Outcome Measures for Individuals With Stroke: Process and Recommendations From the American Physical Therapy Association Neurology Section Task Force; Physical Therapy, Volume 93, Issue 10, 1 Oct. 2013, Pages 1383-1396. Thurman et al.,
Venugopalan et al., Kinect-based Rehabilitation System for Patients with Traumatic Brain Injury. Conf Proc IEEE Eng. Med Biol. Soc. 2013; 4625-28.
Verheyden G et al., The Trunk Impairment Scale: a new tool to measure motor impairment of the trunk after stroke. Clin Rehabil. 2004 May; 18 (3): 326-34.
Verheyden G and Keersten P., Investigating the internal validity of the Trunk Impairment Scale (TIS) using Rasch analysis: the TIS 2.0. Disabil Rehabil. 2010; 32 (25): 2127-37.
Vesterinen H M Currie G L Carter S Systematic review and stratified meta-analysis of the efficacy of RhoA and Rho kinase inhibitors in animal models of ischaemic stroke. Syst Rev. 2013; 2:33.
Williams L S, Measuring quality of life in a way that is meaningful to stroke patients. Neurology. 1999 Nov. 10; 53 (8): 1839-43.
Wilson J T L, Pettigrew L E L, Teasdale G M. Structured interviews for the Glasgow Outcome Scale and the Extended Glasgow Outcome Scale: Guidelines for Their Use. J Neurotrauma 15 (8): 573-85. 1997.

1. A method of treating a patient suffering from chronic effects of brain injury, comprising orally administering to said patient a therapeutically effective amount of a fasudil, hydroxyfasudil, or a pharmaceutically acceptable salt, hydrophobic ion pair or hydrate thereof, wherein said therapeutically effective amount exceeds 60 mg per patient per day, and wherein the brain injury occurred more than three months prior to initiating treatment.

2. The method according to claim 1, wherein fasudil hydrochloride hemihydrate, or molar equivalent thereof, is administered.

3. The method according to claim 1, wherein the brain injury is a traumatic brain injury.

4. The method according to claim 3, wherein the traumatic brain injury resulted from a head contacting a hard surface.

5. The method according to claim 3, wherein the patient exhibits cognitive deficits.

6. The method according to claim 1 wherein the brain injury is a stroke.

7. The method according to claim 6, wherein the stroke is a hemorrhagic stroke.

8. The method according to claim 6, wherein the stroke is a cerebral stroke.

9. The method according to claim 6, wherein the stroke is a cortical stroke.

10. The method according to claim 6, wherein the stroke is a sub-cortical stroke.

11. The method according to claim 6 wherein the stroke is a cerebellar stroke.

12. The method according to claim 7, wherein the stroke is a large vessel stroke.

13. The method according to claim 9, wherein the stroke affects the middle cerebral artery (MCA), the internal carotid artery (ICA), the posterior Inferior cerebellar artery (PICA), and the superior cerebellar artery (SCA).

14. The method according to claim 7, wherein the stroke is a small vessel stroke.

15. The method according to claim 10, wherein the stroke is a lacunar stroke.

16. The method according to claim 7, wherein the hemorrhagic stroke is selected from the group consisting of epidural hematoma, subdural hematoma, subarachnoid hemorrhage (SAH), intraventricular hemorrhage (IVH), hemorrhagic transformation of ischemic stroke (HT), venous hemorrhage from cortical vein or sinus thrombosis, and intracerebral hemorrhage (ICH).

17. The method according to claim 1, wherein the patient does not have hypertension or coronary artery disease.

18. The method according to claim 1, wherein the patient does not have dementia.

19. The method according to claim 6, wherein the stroke is not a sub-cortical stroke or a stroke affecting white matter.

20. The method according to claim 10, wherein the stroke is not a lacunar stroke.

21. The method according to claim 10, wherein the stroke patient does not have Binswanger's disease.

22. The method according to claim 1, wherein fasudil hydrochloride hemihydrate is administered to the patient at a total dose of 70 to 240 mg per patient per day.

23. The method according to claim 1 wherein the fasudil hydrochloride hemihydrate is administered to the patient at a total dose of 90 to 180 mg per patient per day.
